(19) United States
(12) Patent Application Publication
(10) Pub. No.: US 2023/0225819 A1
(43) Pub. Date: Jul. 20, 2023

(54) Auto-Coupling Body Waste Containment Apparatus

(71) Applicant: Emily Molina, Runge, TX (US)

(72) Inventor: Emily Molina, Runge, TX (US)

(21) Appl. No.: 18/154,606

(22) Filed: Jan. 13, 2023

Related U.S. Application Data
(60) Provisional application No. 63/299,444, filed on Jan. 14, 2022.

(51) Int. Cl. A61B 90/00

(52) U.S. Cl. CPC A61B 90/05

(57) ABSTRACT

Apparatus and associated methods relate to an auto-coupling body waste containment apparatus (ABWCA). In an illustrative example, the ABWCA may include a dome container that may be placed over a patient's body portion. The ABWCA includes a self-supported wall defining a cavity. One or more access apertures may, for example, be provided in the wall. A physician's hands may, for example, be inserted along intersecting axes through a second and a second access aperture of the access apertures. The hands may, for example, engage a bottom rim (e.g., a rim) to retain a first aperture of the ABWCA over a body portion of a patient during an operation such that the container is automatically coupled to the body portion by the user's hands. The ABWCA may, for example, advantageously prevent body fluid of the patient from spraying on the physician when providing medical treatment to a patient's body.

### CROSS-REFERENCE TO RELATED APPLICATIONS

This application is a non-provisional application and claims the benefit of provisional U.S. Application Ser. No. 63/299,444, titled “The Dome,” filed by Emily Molina on Jan. 14, 2022.

### TECHNICAL FIELD

Various embodiments relate generally to medical treatments.

### BACKGROUND

Surgical excisions of cysts may, for example, include a procedure to remove the sac and its contents. By way of example and not limitation, three general approaches may be used to remove a cyst: a traditional wide excision, a minimal excision, or a punch biopsy excision. A common surgical procedure for cyst removal may involve using a numbing agent (e.g., a local anesthetic) around the cyst. A scalpel may, for example, be used to open the lesion (e.g., with a single cut bisecting the cyst and/or an oval cut on both sides of the cyst).

In some cases (e.g., if the cyst is small), it may be lanced (e.g., instead of cutting it). The person performing the surgery may, for example, squeeze out the cyst contents. The surgeon may use an instrument such as blunt-headed scissors to hold the incision wide open while using fingers or forceps to try to remove the cyst wall intact.

Acne, also known as acne vulgaris, includes a skin condition that occurs when dead skins and oils from the skin clog hair follicles. Typical features include black heads, white heads, pimples, oily skin and/or possible scarring. Areas of the skin primarily affected with a relatively high number of oil glands include the face, chest, and back. Acne may lead to cysts. In some cases, non-cyst features (e.g., pimples) may be removed.

### SUMMARY

Apparatus and associated methods relate to an auto-coupling body waste containment apparatus (ABWCA). In an illustrative example, the ABWCA may include a dome container that may be placed over a patient's body portion. The ABWCA includes a self-supported wall defining a cavity. One or more access apertures may, for example, be provided in the wall. A physician's hands may, for example, be inserted along intersecting axes through a second and a second access aperture of the access apertures. The hands may, for example, engage a bottom rim (e.g., a lip) to retain a first aperture of the ABWCA over a body portion of a patient during an operation such that the container is automatically coupled to the body portion by the user's hands. The ABWCA may, for example, advantageously prevent body fluid of the patient from spraying on the physician when providing medical treatment to a patient's body.

Various embodiments may achieve one or more advantages. For example, some embodiments may be configured to advantageously couple multiple ABWCA together. Such embodiments may, for example, advantageously create an enclosure for retaining body waste (e.g., nail clippings).

Some embodiments may, for example, be configured with a rotatable ABWCA. Such embodiments may, for example, advantageously enable the ABWCA to be positioned over a body portion and access apertures to be selectively oriented in a desired configuration.

Some embodiments may, for example, include a removable concentric sealing layer(s). Such embodiments may, for example, advantageously provide sealing (e.g., fluid sealing) to a target body portion.

Some embodiments may, for example, advantageously provide removable attachments.

Some embodiments may, for example, advantageously be configured to enable access of medical tools to be used by a physician within the ABWCA.

For example, some embodiments may include a user-manipulable ABWCA. The base of the cavity may, for example, be open such that the ABWCA can be placed over a body portion of a patient during a surgical operation. One or more apertures in the wall of the cavity are provided such that the base of the cavity can be sealingly pressed against the patient's body portion, while accessing the isolated body portion through one or more of the apertures. For example, a user's fingers and/or hand can be inserted through an aperture and pressed down. A portion of the wall below the aperture may be pressed down to maintain the sealing contact of the ABWCA against the body. Fingers and/or surgical instruments (e.g., needle, syringe, scalpel) can be operated in the cavity through the access apertures in the ABWCA.

A portion of the wall of the ABWCA may be transparent. For example, the wall may be transparent for visibility of the surgical operation. The wall may be provided with one or more transparent windows for visibility. The wall may, for example, be substantially rigid. The wall may, for example, be flexible (e.g., silicone) to allow conforming of the wall to a body portion of a user.

In some embodiments, the apertures in the ABWCA may be selectively operable between a closed position and an open position. For example, a user may operate a flap to close the aperture to prevent splashing of fluids (e.g., medications, injectables, body fluid, blood, pus) through the aperture. A user may operate the flap to open the aperture to access the body portion isolated in the cavity

In some embodiments, a self-sealing slit and/or flap may be provided in the wall of the ABWCA. For example, the slit may have abutting edges of flexible material (e.g., silicone). A tool (e.g., scalpel, needle) may be inserted into the cavity through the self-sealing slit. The edges of the slit may fluidly seal around the tool and/or to each other upon removal of the tool.

In some embodiments, a sealing layer may be provided on the base of the ABWCA. For example, a sealing layer may be provided around a periphery (e.g., rim) of the base of the cavity. The sealing layer may fluidly seal the base of the ABWCA to the skin of the patient to fluidly isolate the body portion of the patient within the cavity from an external environment. The sealing layer may, for example, include adhesive (e.g., hydrogel, double-sided tape). The sealing layer may include a dry adhesive (e.g., setae, silicone). In some embodiments, a sealing layer may be pre-serrated to allow multiple sizes of apertures to be formed. For example, the sealing layer may adhere the ABWCA to a portion of a patient's body for hands-free manipulation and/or fluid isolation on an irregular shaped body portion (e.g., curvature of head).

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary auto-coupling body waste containment apparatus (ABWCA) employed in an illustrative use-case scenario.

FIG. 2 depicts an illustrative use-case scenario of a physician's hands position while employing an exemplary ABWCA.

FIG. 3 depicts an exemplary illustrative embodiment of a first pair of perforations and a second pair of perforations of an exemplary ABWCA along with a coupling of a first ABWCA.

FIG. 4 depicts an exemplary use-case scenario of a physician using a low-profile insertion of an apparatus using an exemplary ABWCA into a treatment site of a portion of a patient's body.

FIG. 5 depicts an exemplary ABWCA that includes a rotatable base.

FIG. 6 depicts an exemplary ABWCA including a sealing layer, an adhesive, and concentric pre-serrations.

FIG. 7 depicts several exemplary ABWCA embodiments such that the second and second access apertures are configured with shapes and sized apertures.

FIG. 8 depicts several exemplary ABWCA embodiments such as a slit cover, a flap cover, and a ABWCA cap.

Like reference symbols in the various drawings indicate like elements.

### DETAILED DESCRIPTION OF ILLUSTRATIVE EMBODIMENTS

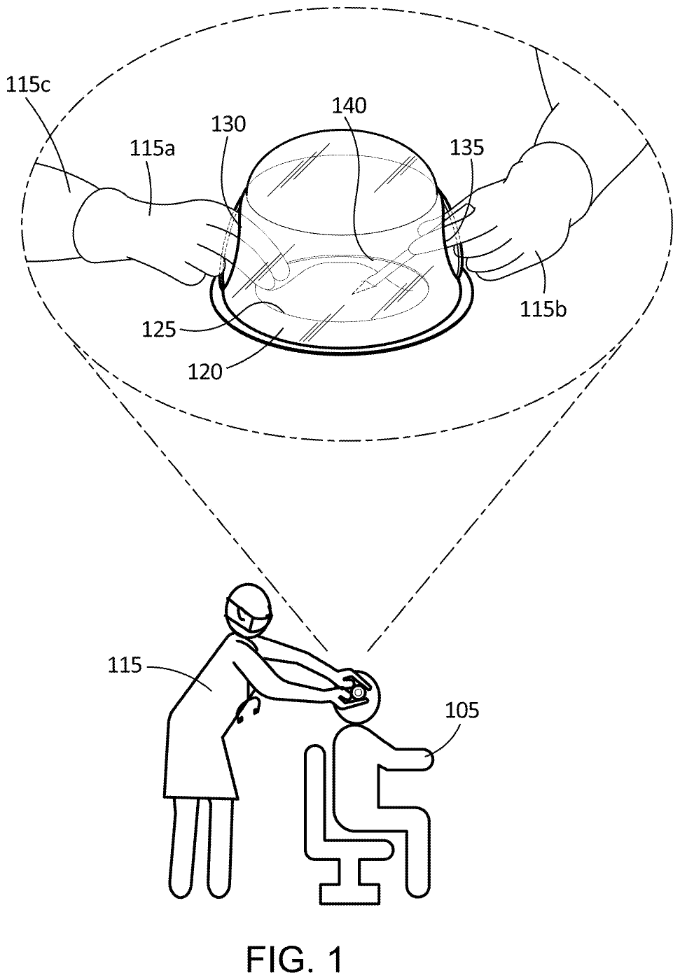

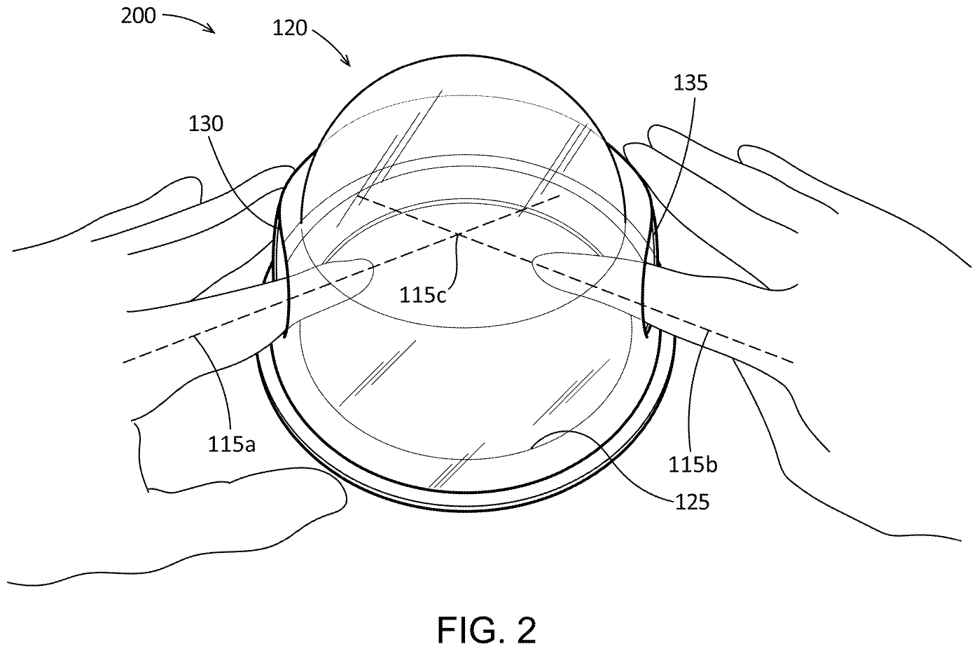

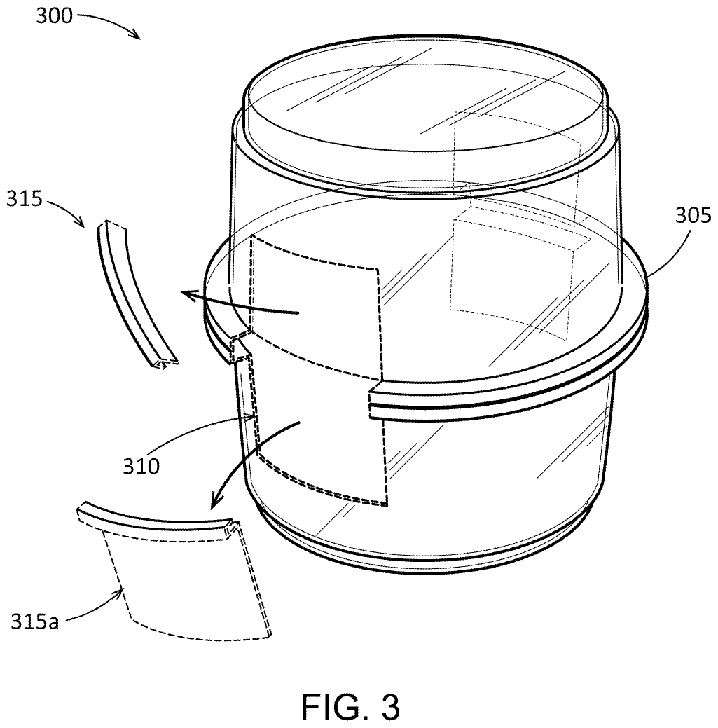

To aid understanding, this document is organized as follows. First, to help introduce discussion of various embodiments, an exemplary ABWCA is introduced with reference to FIGS. 1-2. Second, that introduction leads into a description with reference to FIGS. 3-8 of some exemplary embodiments of the auto-coupling body waste containment apparatus (ABWCA).

FIG. 1 depicts an exemplary ABWCA employed in an illustrative use-case scenario 100. The illustrative use-case scenario 100 includes a patient 105. The patient 105 has a body portion 110. The body portion may, for example, include a cyst. The body portion 110 is being treated by a physician 115. The physician 115 is using a ABWCA 120. The physician 115 uses their hand 115a to place the ABWCA 120. The ABWCA 120 includes a self-supported wall defining a cavity. The ABWCA includes a first aperture. The first aperture is configured to be an opening to encase the body portion being treated. A portion of the user's hands may be used to treat a body portion. The portion of the user's hands may, for example, include a finger, two fingers and a thumb, such that the user's hands may perform treatment procedures. The treatment procedures may, for example, include squeezing excess fluid from the cyst after removing the cyst wall.

The ABWCA 120 includes a first aperture 125. The ABWCA may, for example, include a dome. The first aperture 125 is centered over the body portion 110. The first aperture 125 is enclosed by a bottom lip 126 of the self-supported wall of the ABWCA. The bottom lip may, for example, allow a physician to use their hands to retain the bottom lip of the self-supported wall to the body portion. The physician may, for example, place a portion of their hands through the first access aperture to treat the body portion. The ABWCA 120 includes a first access aperture 130. The physician may, for example, place a portion of one of their hands through the first access aperture to treat the body portion. The ABWCA 120 includes a second access aperture 135. The physician may, for example, place a portion of one of their hands through the second access aperture to treat the body portion. The portion of their hands may, for example, include fingers. The physician may, for example, use both of their hands to hold the ABWCA over a body portion while using their palms to hold the first aperture centered over the body portion while placing their hands through the second and second access aperture to treat the body portion.

The physician 115 is holding an apparatus 140 to treat the body portion. The apparatus may, for example, include a scalpel. The physician may, for example, use a variety of medical apparatuses in conjunction with the ABWCA to treat the body portion. The ABWCA may, for example, shield the physician from body fluids that spray from the treatment of the body portion. The internal pressure of the ABWCA may, for example, be ambient. The ambient pressure may, for example, advantageously prevent unwanted and/or premature disruption the body portion, such as rupturing a cyst's wall as a physician is removing the cyst.

FIG. 2 depicts an illustrative use-case scenario 200 of a of a physician's hands position is being while employing an exemplary ABWCA. The illustrative use-case scenario 200 includes the ABWCA 120. The illustrative use-case scenario 200 includes a physician's hands holding a ABWCA over a body portion. The ABWCA is being held by a portion of the physician's hands (e.g., the physician's palms) while a portion of the physician's hands (e.g., some of the physician's fingers and knuckles) are being placed into the second and second access aperture respectively. The illustrative use-case scenario 200 includes a path of a portion of the user's hands 115b. The illustrative use-case scenario 200 includes a first aperture 125. The illustrative use-case scenario 200 includes a first access aperture 130. The illustrative use-case scenario 200 includes a second access aperture 135. In a two-handed mode, one of each path of a portion of the user's hand 115b is aligned to go through the first access aperture 130. In a two-handed mode, one of each path of a portion of the user's hand 115b is aligned to go through the second access aperture 135. In a two-handed mode, each of the paths of a portion of the user's hand 115b intersect at an intersection 115c in the ABWCA. The intersection may, for example, be located on the treatment area of the patient's body.

FIG. 3 depicts an exemplary illustrative embodiment 300 of a first pair of perforations and a second pair of perforations of a ABWCA along with a coupling of a first ABWCA. The exemplary illustrative embodiment 300 includes a bottom lip 305 of the ABWCA. The user's hands may, for example, retain on a bottom lip of the wall to the body portion. The exemplary illustrative embodiment 300 includes a first pair of perforations 310. The exemplary illustrative embodiment 300 includes detachable attachments 315. The detachable attachments may, for example, include a first cover, a second cover, and/or a portion of a bottom lip.

The exemplary illustrative embodiment 300 includes a second pair of perforations 310a. The bottom lip 305 includes a portion of the bottom lip 315a. The second pair of perforations 310a allows the decoupling of a portion of a bottom lip 315a. A first set of the second pair of perforations may, for example, enclose the first portion of the bottom lip. For example, a user may split the first set of the second pair of perforations of the first portion of the bottom lip to decouple the first portion of the bottom lip from the bottom lip of the ABWCA. For example, a second set of perforations of the second pair of perforations enclose a second portion of the bottom lip. A user may, for example, split the second set of the second pair of perforations of the second portion of the bottom lip to decouple the second portion of the bottom lip from the bottom lip of the ABWCA.

The exemplary illustrative embodiment 300 includes a first ABWCA. The exemplary illustrative embodiment 300 includes a second ABWCA. The first ABWCA and second ABWCA may be coupled to form a container. The container may, for example, be used as a storage container for medical waste. The medical waste may, for example, include nails, blood, pus, skin, used bandages, and/or used wipes. The container may, for example, be used during surgery to store hold fluids, bandages, medical apparatuses, and/or wipes.

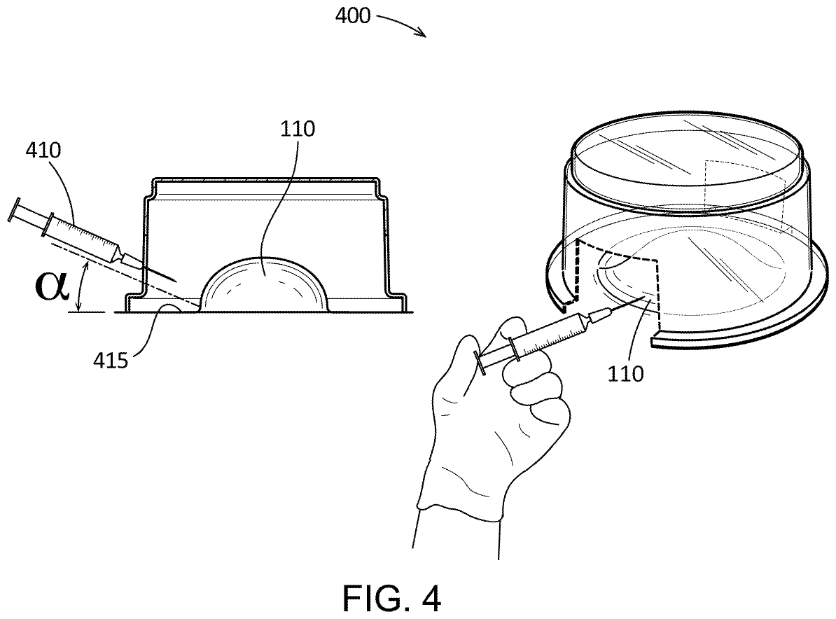

FIG. 4 depicts an exemplary use-case scenario 400 of a physician using a low-profile insertion of an apparatus using a ABWCA into a treatment site of a portion of a patient's body. The exemplary use-case scenario 400 includes a body portion 110. The exemplary use-case scenario 400 includes an apparatus 410. The apparatus may, for example, include a needle. The apparatus 410 is being used to treat the body portion 110. The apparatus 410 is at an angle 415 from surface of the skin. The ABWCA may, for example, allow for a low-profile insertion while the ABWCA shields the user from any possible fluid spray from the body portion being treated.

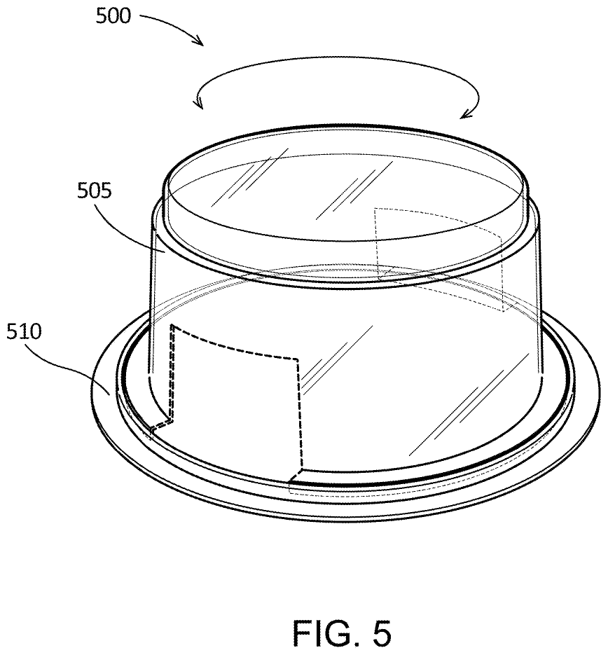

FIG. 5 depicts an exemplary ABWCA 500 that includes a rotatable base. The exemplary ABWCA 500 includes a self-supported wall 505. The self-supported wall 505 is rotatably coupled to a rotatable bottom lip 510. The self-supported wall may, for example, rotate counterclockwise. The self-supported wall may, for example, rotate clockwise. The rotatable bottom lip may, for example, be attached with an adhesive. The adhesive may, for example, keep the rotatable bottom lip in place such that the self-supported wall may rotate. The rotation may, for example, allow a physician to dynamically treat the body portion while still being shielded from body fluid of the patient.

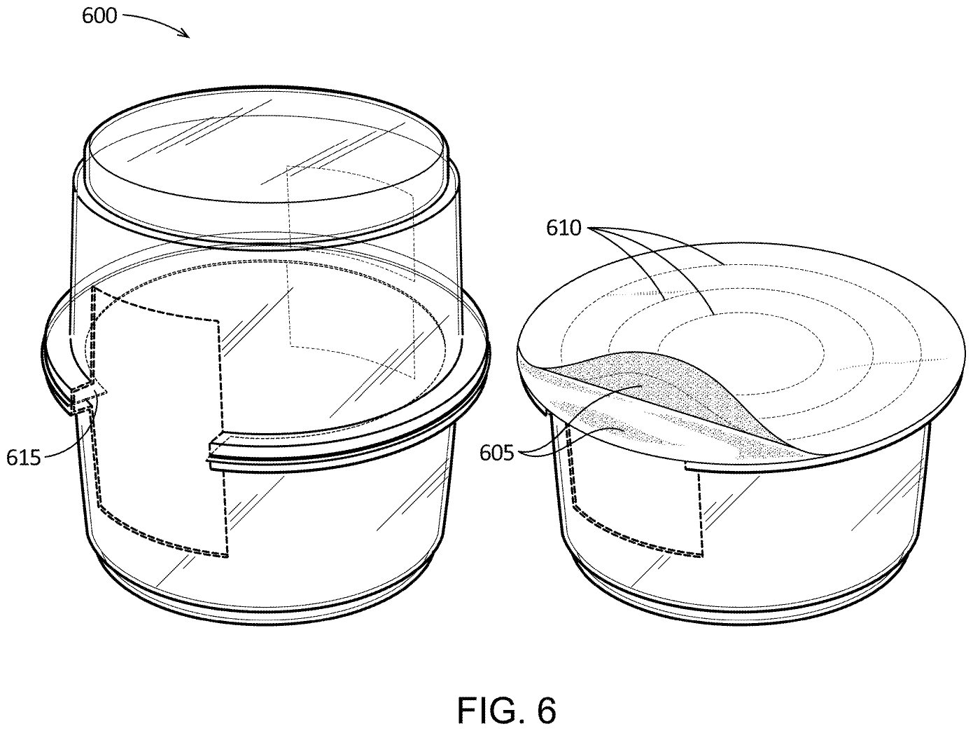

FIG. 6 depicts an exemplary ABWCA 600 including a sealing layer with an adhesive, and concentric pre-serrations. The exemplary ABWCA 600 includes a sealing layer 605 with adhesive and a protective liner. The protective liner may be removed (e.g., as shown on the right) to expose the adhesive 615. The exemplary ABWCA 600 includes a sealing layer 610. The sealing layer is provided on the base of the ABWCA such that a sealing layer may be provided around a periphery of the base of the cavity. The sealing layer may, for example, include gauze. The sealing layer may, for example, include absorbent material. The absorbent material may, for example, absorb fluid while being placed on the periphery. The sealing layer may fluidly seal the base of the ABWCA to the skin of the patient to fluidly isolate the body portion of the patient within the cavity from an external environment.

The sealing layer 610 includes concentric pre-serrations such that the sealing layer may be configured to encompass a close-fit of a patient's body. The close-fit of a patient's body may, for example, enclose a cyst.

The layers of the sealing layer 610 may, for example, allow fluid isolation on an irregularly shaped body portion. The adhesive 615 may, for example, allow a user to adhere the ABWCA to a portion of a patient's body for hands-free manipulation and fluid isolation on an irregularly shaped body portion.

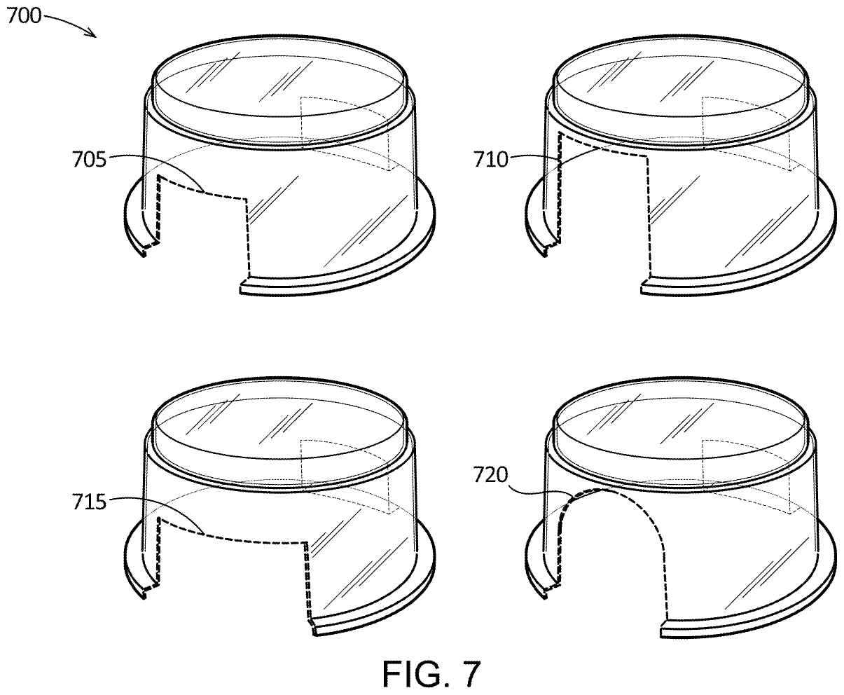

FIG. 7 depicts several exemplary ABWCA embodiments 700 such that the second and second access apertures are configured with shapes and sized apertures. The several exemplary ABWCA embodiments 700 includes a rectangular aperture 705. The several exemplary ABWCA embodiments 700 includes a longitudinally extended aperture 710. The several exemplary ABWCA embodiments 700 includes a latitudinally extended aperture 715. The several exemplary ABWCA embodiments 700 includes an oval shaped aperture 720.

The ABWCA may, for example, have different sizes. The bottom rim of the ABWCA may, for example, be 4″ in diameter. The bottom rim of the ABWCA may rim of the first aperture may, for example, be 6″ in diameter. The bottom rim of the ABWCA may rim of the first aperture may, for example, be 8″ in diameter. The ABWCA may, for example, be sold in sets, such that the ABWCA may come in 1″-6″ in 0.5″ increments.

The ABWCA may, for example, have different shapes. The ABWCA may, for example, be oval shaped. The ABWCA may, for example, be rectangular shaped. The ABWCA may, for example, be circularly shaped. The ABWCA may, for example, be triangularly shaped. The ABWCA may, for example, be a pyramid shaped. The ABWCA may, for example, be hexagonal shaped. The ABWCA may, for example, be octangular shaped. The ABWCA may, for example, be square shaped. The ABWCA may, for example, be pentagonal shaped.

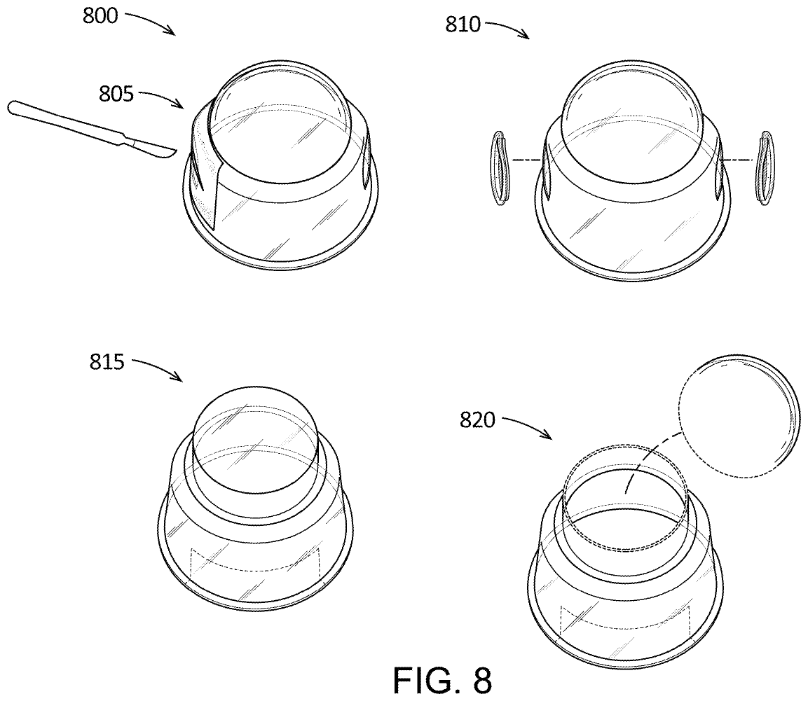

FIG. 8 depicts several exemplary ABWCA embodiments 800 such as a slit cover, a flap cover, and a ABWCA cap. The several exemplary ABWCA embodiments 800 includes a self-sealing slit 805. The self-sealing slit may, for example, have abutting edges of flexible material such that a tool may be inserted into the cavity. The self-sealing slit and the edges of the slit may fluidly seal a treatment area around the tool.

The several exemplary ABWCA embodiments 800 includes a flap 810. The apertures in the ABWCA may be selectively operable between a closed position and an open position. For example, a user in an open mode may operate the flap to close the aperture to prevent splashing of fluids through the aperture. For example, a user in a closed mode a user may operate the flap to open the aperture to access the body portion isolated in the cavity. The flap may, for example, be detachable. The flap may, for example, be attached by at least one hinge to the ABWCA.

The several exemplary ABWCA embodiments 800 includes a closed mode ABWCA 815. The several exemplary ABWCA embodiments 800 includes an open mode ABWCA 820. For example, a in a close mode ABWCA may operate to treat a patient from the second and the second access aperture. In an open mode, a physician could open the ABWCA to have access to the body portion from above. The open ABWCA mode includes a top portion of the ABWCA that may be decoupled from the closed mode ABWCA. The decoupling may, for example, be from a set of perforations proximal to the top portion of the ABWCA distal to the first aperture. The decoupling may, for example, allow the user to remove the top portion of the ABWCA to have unobstructed above access to the body portion which the ABWCA is centered on. The physician may, for example, prefer to remove the top portion of the ABWCA compared to removing the ABWCA, because of desired protection against spills of body fluids. The physician may, for example, prefer to remove the top portion of the ABWCA compared to removing the ABWCA, because the ABWCA may be coupled with adhesive to the patient.

Although various embodiments have been described with reference to the figures, other embodiments are possible.

Although an exemplary system has been described with reference to FIG. 1-8, other implementations may be deployed in other industrial, scientific, medical, commercial, and/or residential applications. The ABWCA may, for example, be used in medical facilities and offices. The ABWCA may, for example, be used at a dermatologist's office. The ABWCA may, for example, be available at sale at super markets in a cosmetic aisle for at home treatment. The ABWCA may, for example, be included in cosmetic kits.

A kit may, for example, include one or more ABWCAs (e.g., 2 ABWCAs). The kit may, for example, include an adhesive layer (e.g., on one or more of the ABWCAs, as a separate component that may be selectively applied to one or more of the ABWCAs). The kit may, for example, include a wipe (e.g., sanitizing wipe). The kit may, for example, include a disposal container (e.g., bag, sealing layer). The kit may, for example, include one or more accessories and/or tools (e.g., cap, base, scalpel, syringe, needle, lancet).

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. An auto-coupling body waste containment apparatus (ABWCA) comprising:
a self-supported wall defining a cavity having a first aperture configured to be placed over a body portion;
a first access aperture and a second access aperture, each configured to receive a corresponding object inserted along intersecting paths extending through the self-supported wall; and,
a portion of the self-supported wall passing between the first aperture and each of the first access aperture and the second access aperture to form a bottom rim configured such that, when the corresponding portion of hand of a user are inserted through the first access aperture and the second access aperture, the hands engage the bottom rim such that at least a portion of a periphery of the first aperture is retained against the body portion.
a first pair of perforations, wherein a first set of the first pair of perforations enclose a first cover of the first access aperture such that the first cover may be removed to form the first access aperture by splitting the first set of perforations, and a second set of perforations of the first pair of perforations enclose a second cover of the second access aperture such that the second cover may be removed to form the second access aperture by splitting the second set of perforations;
wherein an internal pressure within the cavity is ambient.

2. The apparatus of claim 1, further comprising a second pair of perforations, wherein a first set of the second pair of perforations enclose a first portion of the bottom rim such that a user may split the first set of the second pair of perforations of the first portion of the bottom rim to decouple the first portion of the bottom rim from the bottom rim, and a second set of perforations of the second pair of perforations enclose a second portion of the bottom rim such that a user may split the second set of the second pair of perforations of the second portion of the bottom rim to decouple the second portion of the bottom rim from the bottom rim.

3. The apparatus of claim 2, further comprising a rotation mechanism such that the self-supported wall is configured to be selectively rotated relative to the bottom rim.

4. The apparatus of claim 2 being a first container, and a kit comprising the first container and a second container comprising a first access aperture into a second cavity, the bottom rim being configured to releasably couple a periphery of the first aperture to a periphery of the first access aperture to form a unified container.

5. The apparatus of claim 3, further comprising a flap over at least one of the first access aperture and the second access aperture, such that the flap may be selectively operable between:
a closed position configured to prevent splashing of fluids through the corresponding aperture; and,
an open position configured to permit access to the body portion isolated in the cavity.

6. The apparatus of claim 4, further comprising a sealing layer provided on a base of the cavity such that a sealing layer may be provided around a periphery of the base, wherein the sealing layer may fluidly seal the base to fluidly isolate the body portion within the cavity from an external environment.

7. A container comprising:
a self-supported wall defining a cavity having a first aperture configured to be placed over a body portion;
a first access aperture and a second access aperture, each configured to receive a corresponding object inserted along intersecting paths extending through the self-supported wall; and,
a portion of the self-supported wall passing between the first aperture and each of the first access aperture and the second access aperture to form a bottom rim configured such that, when the corresponding hands of a user are inserted through the first access aperture and the second access aperture, the hands engage the bottom rim such that at least a portion of a periphery of the first aperture is retained against the body portion.

8. The container of claim 7, wherein an internal pressure within the cavity is ambient.

9. The container of claim 8, further comprising a first pair of perforations, wherein a first set of the first pair of perforations enclose a first cover of the first access aperture such that the first cover may be removed to form the first access aperture by splitting the first set of perforations, and a second set of perforations of the first pair of perforations enclose a second cover of the second access aperture such that the second cover may be removed to form the second access aperture by splitting the second set of perforations.

10. The container of claim 9, further comprising a top portion that may be decoupled along a set of perforations proximal to the top portion and distal to the first aperture such that the top portion may be removed to have unobstructed above access to the body portion.

11. The container of claim 10, further comprising a rotation mechanism such that the self-supported wall is configured to be selectively rotated relative to the bottom rim.

12. The container of claim 9, further comprising a second pair of perforations, wherein a first set of the second pair of perforations enclose a first portion of the bottom rim such that a user may split the first set of the second pair of perforations of the first portion of the bottom rim to decouple the first portion of the bottom rim from the bottom rim, and a second set of perforations of the second pair of perforations enclose a second portion of the bottom rim such that a user may split the second set of the second pair of perforations of the second portion of the bottom rim to decouple the second portion of the bottom rim from the bottom rim.

13. The container of claim 12 being a first container, and a kit comprising the first container and a second container comprising a first access aperture into a second cavity, the bottom rim being configured to releasably couple a periphery of the first aperture to a periphery of the first access aperture to form a unified container.

14. The container of claim 13, further comprising a flap over at least one of the first access aperture and the second access aperture, such that the flap may be selectively operable between:
a closed position configured to prevent splashing of fluids through the corresponding aperture; and,
an open position configured to permit access to the body portion isolated in the cavity.

15. The container of claim 14, further comprising, a self-sealing member having a self-sealing slit defined by abutting edges of flexible material and configured such that a tool may be inserted into the cavity through the self-sealing slit and the edges of the slit may fluidly seal a treatment area around the tool.

16. The container of claim 15, further comprising a sealing layer provided on a base of the cavity such that a sealing layer may be provided around a periphery of the base, wherein the sealing layer may fluidly seal the base to fluidly isolate the body portion within the cavity from an external environment.

17. The container of claim 16, wherein the sealing layer includes an adhesive such that the sealing layer may adhere the bottom rim to a portion of a patient's body for hands-free manipulation and fluid isolation on an irregularly-shaped body portion.

18. The container of claim 17, wherein the sealing layer includes pre-serrations define a plurality of user-selectable predetermined geometries of the first aperture.

19. The container of claim 18, wherein the object comprises at least one portion of a hand of a user.

20. The container of claim 19, wherein the object comprises at least one portion of a tool.
